(19) United States
(12) Patent Application Publication
(10) Pub. No.: US 2011/0088463 A1
(43) Pub. Date: Apr. 21, 2011

(54) PERFORMANCE ENHANCEMENT MEASUREMENT DEVICE FOR JOINTS

(76) Inventors: Ronald S. Shapiro, Toledo, OH (US); David B. Sutton, Monroe, MI (US); Richard C. Dunlap, Holland, OH (US)

(21) Appl. No.: 12/904,408

(22) Filed: Oct. 14, 2010

Related U.S. Application Data
(60) Provisional application No. 61/252,362, filed on Oct. 16, 2009.

(51) Int. Cl. A61B 5/11

(52) U.S. Cl. 73/172

(57) ABSTRACT

A measurement device includes a housing, a sensor disposed in the housing to measure a force exerted on the housing and generate a signal representing the force exerted on the housing, and a digital display for receiving the signal from the sensor and presenting a visual output to a user based upon the force exerted on the housing.

### CROSS-REFERENCE TO RELATED APPLICATION

This application claims the benefit of U.S. Provisional Patent Application No. 61/252,362 filed on Oct. 16, 2009, the entire disclosure of which is incorporated herein by reference.

### FIELD OF THE INVENTION

The present invention is generally related to a pain and performance assessment device. More particularly, the invention is directed to a measurement device and a method for assessing and quantifying a pain and a performance level of a user.

### BACKGROUND OF THE INVENTION

The sensation of pain means different things to different people. To date, pain has been extraordinarily difficult to quantify without the use of highly sophisticated and expensive medical diagnostic equipment. Often, as a simple means to quantify pain in a clinical setting, the patient is given a scale with an associative face and a number to illustrate the varying degrees of pain. Still, the conventional pain scale is inconsistent and insufficient to properly describe or quantify pain and monitor pain remediation, particularly as it relates to their ability to perform at an enhanced level.

It would be desirable to develop a performance enhancement measurement device to readily assess and quantify a pain and a performance level of a user, wherein the measurement device is usable with any joints on a body of the user.

### SUMMARY OF THE INVENTION

Concordant and consistent with the present invention, a performance enhancement measurement device to readily assess and quantify a pain and a performance level of a user, wherein the measurement device is usable with any joints on a body of the user, has surprisingly been discovered.

The performance enhancement measurement device provides an individual with a “pain number” or “performance number” associated with an affected joint (knee, elbow, wrist, hand, etc.) prior to therapy and then provides a post treatment number associated with the same affected joint in order to quantify and measure the improvement and increased mobility. Since pain is so subjective and variable between individuals, a more precise measurement of change in pain and performance with treatment as well as identifying a significant “pain number” would offer new dimensions in assessment in many disciplines. Additionally, the measurement device provides a means for building strength and quantifying a strength performance.

In one embodiment, a measurement device comprises a compressible housing, a pressure sensor disposed in the housing to measure a pressure exerted on the housing and generate a signal representing the pressure measurement, and a display for receiving the signal from the pressure sensor and presenting a visual output to a user based upon the pressure measurement.

Methods for using a measurement device are also disclosed.

One method comprises the steps of: a) providing the measurement device, wherein the measurement device further comprises: a housing; a sensor disposed in the housing to measure a force exerted on the housing and generate a pressure signal representing the force exerted on the housing; and a digital display for receiving the pressure signal from the sensor and presenting a visual output to a user based upon the force exerted on the housing; b) disposing the measurement device adjacent a portion of a body of a user; and c) exerting a force on the housing of measurement device using the body of the user, wherein the display presents the visual output representing the force exerted on the housing.

Another method comprises the steps of: a) providing the measurement device, wherein the measurement device further comprises: a housing; a sensor disposed in the housing to measure a force exerted on the housing and generate a pressure signal representing the force exerted on the housing; and a digital display for receiving the pressure signal from the sensor and presenting a visual output to a user based upon the force exerted on the housing; b) disposing the measurement device adjacent a portion of a body of a user; and c) exerting a force on the housing of measurement device using the body of the user, wherein the display presents the visual output representing the force exerted on the housing.

### BRIEF DESCRIPTION OF THE DRAWINGS

The above, as well as other advantages of the present invention, will become readily apparent to those skilled in the art from the following detailed description of the preferred embodiment when considered in the light of the accompanying drawings in which:

FIG. 1 is a front elevational view of a measurement device according to an embodiment of the present invention; and

FIG. 2 is a schematic block diagram of the measurement device of FIG. 1;

FIG. 3 is a perspective view of the measurement device of FIG. 1 in a palm of a hand of a user;

FIG. 4 is a perspective view of the measurement device of FIG. 1 disposed adjacent a knee joint of a user; and

FIG. 5 is a schematic flow diagram of a method for using the measurement device of FIG. 1.

### DETAILED DESCRIPTION OF EXEMPLARY EMBODIMENTS OF THE INVENTION

The following detailed description and appended drawings describe and illustrate various embodiments of the invention. The description and drawings serve to enable one skilled in the art to make and use the invention, and are not intended to limit the scope of the invention in any manner. In respect of the methods disclosed, the steps presented are exemplary in nature, and thus, the order of the steps is not necessary or critical.

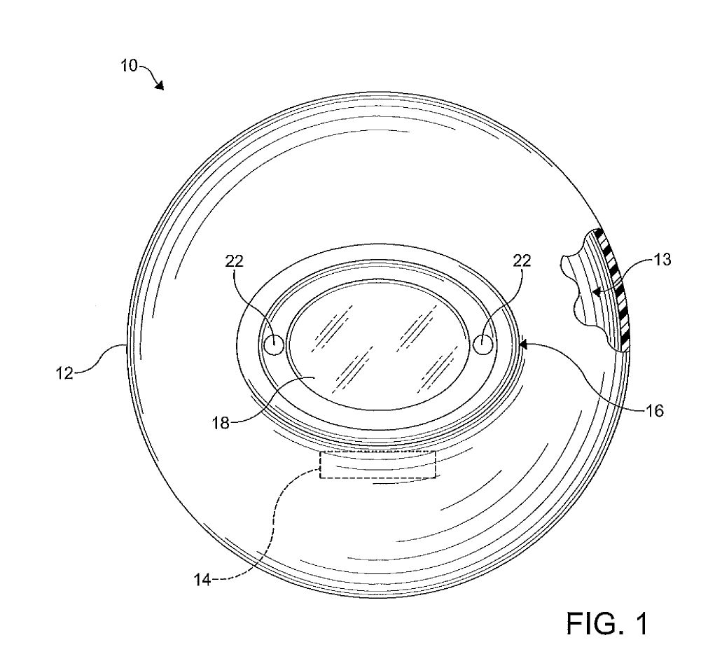

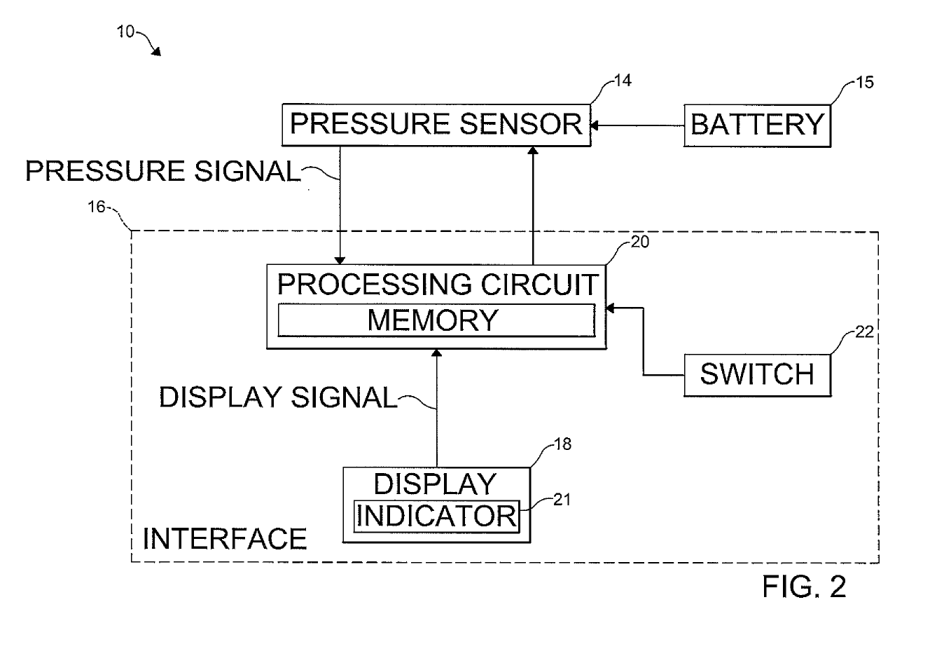

FIGS. 1-2 illustrate a performance enhancement measurement device 10 for assessing a pain and/or performance level of a user. As shown, the measurement device 10 includes a housing 12, a pressure sensor 14, and a user interface 16.

The housing 12 is typically formed from a compressible material, although other materials can be used. In the embodiment shown, the housing 12 has a spherical shape, wherein an inside surface of the housing 12 defines a pressure cavity 13 therein. It is understood that the housing 12 and the pressure cavity 13 may have any size and shape. It is further understood that the housing 12 may be unitary or formed from a plurality of components to define the pressure cavity 13. In certain embodiments, the housing 12 includes a pressure port (not shown) for introducing or releasing a fluid from the pressure cavity 13.

The pressure sensor 14 is disposed in the pressure cavity and adapted to measure at least one of an absolute pressure and a differential pressure of the pressure cavity. As a non-limiting example, the pressure sensor 14 is one of an NPA series sensor and an NPX-SPI series sensor manufactured by GE® which incorporate an application-specific integrated circuit (ASIC) to convert an analog pressure measurement to a digital format that can be directly driven to an output such as a display panel, for example. It is understood that other sensors and strain gauges may be used. As a further example, an electromechanical pressure sensor may be disposed in the pressure cavity to directly measure a compression force applied to the housing 12.

A battery 15 is in electrical communication with the pressure sensor 14 to provide an electric power to the pressure sensor 14 for operation thereof. It is understood that the battery 15 can provide electrical power to other components of the measurement device 10. As a non-limiting example the battery 15 is a button cell battery. In certain embodiments, the battery 15 is disposed in a chamber (not shown) that is isolated from the pressure cavity, wherein the housing 12 includes a removable panel for providing selective access to the battery 15 without disturbing a pressure measurement of the pressure cavity.

The interface 16 includes a display 18, a processing circuit 20, and at least one switch 22 or button. The display 18 shown is a liquid crystal display. However, other displays may be used. The display 18 is disposed on an exterior surface of the housing 12 and in electrical communication with the processing circuit 20 to drive the visual output presented thereon. It is understood that the battery 15 may be in electrical communication with the display 18 to provide electric power thereto. In certain embodiments, the display 18 is disposed in the pressure cavity 13 and the housing 12 includes a generally transparent window (not shown) for viewing the display 18 therethrough.

The processing circuit 20 includes a driver for controlling the visual output presented on the display 18. The processing circuit 20 also includes a processor to receive a pressure signal representing a measurement of the pressure sensor 14 and convert the pressure signal into a display signal representing an indicator 21 (e.g. a performance indicator or a pain indicator). It is understood that the indicator may be any symbol or alphanumeric character for conveying a feedback to the user based upon a pressure applied to the housing 12. It is further understood that the pressure sensor 14 may include an integrated processing circuit, wherein the processing circuit 20 would not be required.

The switch 22 is disposed on an exterior surface of the housing 12 and in electrical communication with the processing circuit 20 to control a function thereof. For example, the switch 22 may actuate a reset function to clear the display 18 of any visual output. The switch 22 may also control an “ON” and “OFF” state of the display 18 and pressure sensor 14. In certain embodiments, the processing circuit 20 includes a memory device 24, wherein the switch 22 provides control of the information stored to the memory device 24 and retrieved therefrom. As a non-limiting example, the processing circuit 20 is configured to compare a pressure measurement from the pressure sensor 14 to data stored in/on the memory device 24.

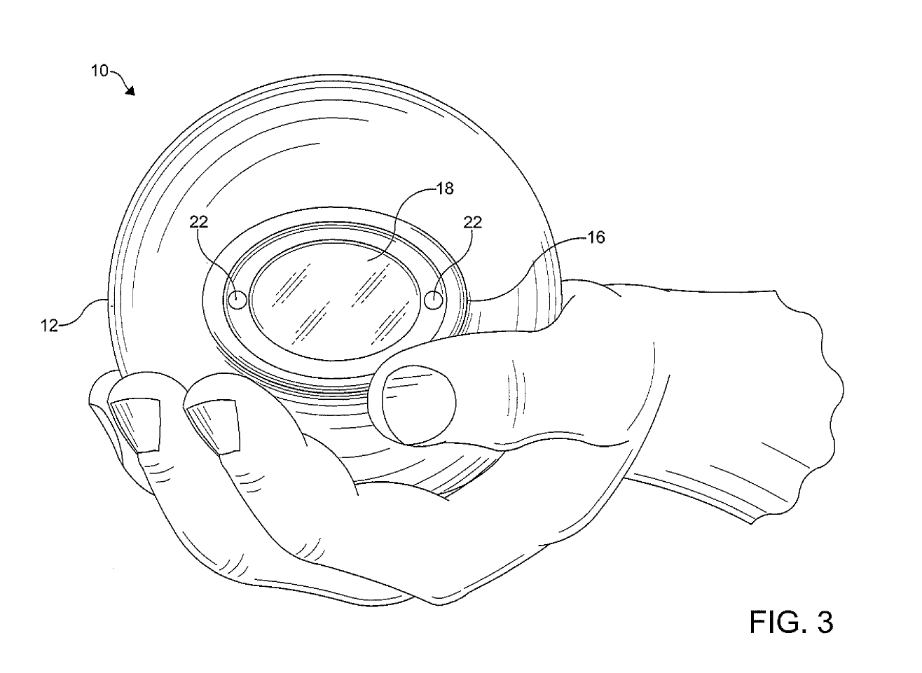

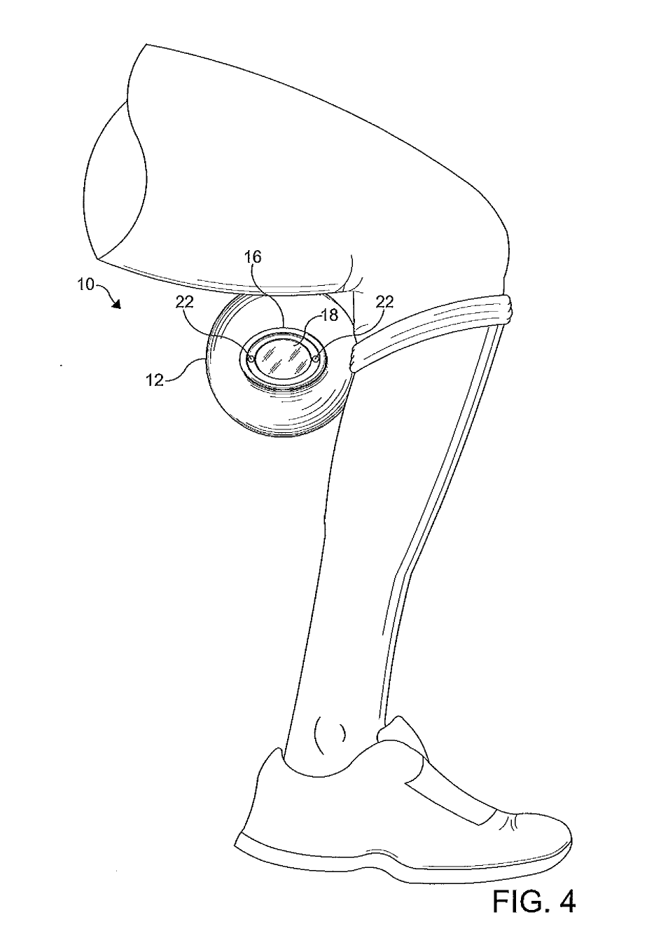

In use, the measurement device 10 is disposed adjacent a joint of a body of the user. As a non-limiting example, a user grasps the measurement device 10 in his/her hand and squeezes until the user has a sensation of pain or achieves a maximum grip or range of motion, as illustrated in FIG. 3. As a further non-limiting example, the measurement device 10 is disposed behind a knee joint of the user, wherein the measurement device 10 is held between a thigh and a calf of the user, as illustrated in FIG. 4.

As the user exerts a force on the measurement device 10, the pressure sensor 14 measures the absolute pressure or a change in pressure of the pressure cavity 13 and transmits the pressure signal to the processing circuit 20. In certain embodiments, the pressure sensor 14 directly measures a force or pressure exerted on the housing 12. The processing circuit 20 converts the pressure signal into a display signal which drives the display 18 to present the visual output (representing the indicator 21).

In certain embodiments, the visual output (e.g. indicator 21) represents a baseline comparison number/indicator (e.g. a “pain number” or a “performance number”). As a non-limiting example the visual output represents a pain indicator, wherein the user exerts a force on the housing 12 until a pain is experienced. As a further non-limiting example, the visual output represents a performance indicator, wherein the user exerts a maximum achievable force on the housing 12 using a body of the user. After reading and recording the visual output (e.g. indicator or number) the user resets the display 18 to zero and subsequently repeats the process for comparison. In this way, the user can quantify an improvement and determine if the therapy or training is reducing a pain or improving a mobility or strength (performance). In certain embodiments, the measurement device 10 includes a data port (e.g. universal serial bus, wireless data connection) to facilitate an extraction of a data such as the performance number, for example.

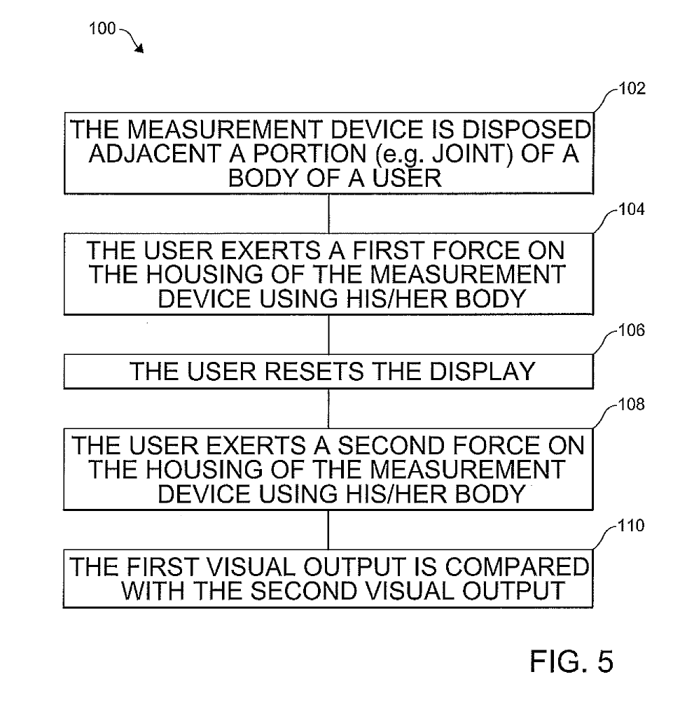

FIG. 5 illustrates a method 100 for using the measurement device 10 according to an embodiment of the present invention. In step 102, the measurement device 10 is disposed adjacent a portion (e.g. joint) of a body of a user. In step 104, the user exerts a first force on the housing 12 of measurement device 10 using the body of the user, wherein the display 18 presents a first visual output representing the pressure measurement resulting from the first force exerted on the housing 12. In step 106, the user resets the display 18. In step 108, the user exerts a second force on the housing 12 of measurement device 10 using the body of the user, wherein the display 18 presents a second visual output representing the pressure measurement resulting from the second force exerted on the housing 12. In step 110, the first visual output is compared (e.g. by the user, other person, or processor, for example) with the second visual output to determine a change between the first force and the second force exerted on the housing 12 of the measurement device 10. In this way, the user can quantify an improvement and determine if the therapy or training is reducing a pain or improving a mobility or strength (performance).

Current methodologies for evaluating pain are imprecise and inconsistent. Evaluating pain and performance with the performance enhancement measurement device 10 produces precise and consistent results. The measurement device 10 provides a real-time aide to enhance a therapy, a rehabilitation, and an overall physical and psychological performance.

From the foregoing description, one ordinarily skilled in the art can easily ascertain the essential characteristics of this invention and, without departing from the spirit and scope thereof, make various changes and modifications to the invention to adapt it to various usages and conditions.

What is claimed is:

1. A measurement device comprising:
a housing;
a sensor disposed in the housing to measure a force exerted on the housing and generate a pressure signal representing the force exerted on the housing; and
a digital display for receiving the pressure signal from the sensor and presenting a visual output to a user based upon the force exerted on the housing.

2. The measurement device according to claim 1, wherein the housing is a compressible housing.

3. The measurement device according to claim 1, wherein the housing has a substantially spherical shape.

4. The measurement device according to claim 1, wherein the housing further includes a pressure cavity disposed therein.

5. The measurement device according to claim 4, wherein the sensor is disposed in the pressure cavity.

6. The measurement device according to claim 1, wherein the sensor is configured to measure an absolute pressure.

7. The measurement device according to claim 1, wherein the sensor is configured to measure a differential pressure.

8. The measurement device according to claim 1, wherein the pressure signal is a digital signal representing an analog measurement of the force exerted on the housing.

9. The measurement device according to claim 1, further comprising a circuit to receive the pressure signal representing the force exerted on the housing and convert the pressure signal into a display signal for controlling the visual output presented on the display.

10. The measurement device according to claim 1, wherein the visual output presented on the display is a pain indicator.

11. The measurement device according to claim 1, wherein the visual output presented on the display is a performance indicator.

12. The measurement device according to claim 1, further comprising a means to reset the visual output presented on the display.

13. The measurement device according to claim 1, further comprising a memory means to store at least a data representing the pressure measurement of the sensor.

14. The measurement device according to claim 1, further comprising a battery in electrical communication with at least the sensor.

15. A method for using a measurement device comprising the steps of:
a) providing the measurement device, wherein the measurement device further comprises:
a housing;
a sensor disposed in the housing to measure a force exerted on the housing and generate a pressure signal representing the force exerted on the housing; and
a digital display for receiving the pressure signal from the sensor and presenting a visual output to a user based upon the force exerted on the housing;
b) disposing the measurement device adjacent a portion of a body of a user; and
c) exerting a force on the housing of measurement device using the body of the user, wherein the display presents the visual output representing the force exerted on the housing.

16. The method according to claim 15, wherein the visual output presented on the display is a pain indicator.

17. The method according to claim 15, wherein the visual output presented on the display is a performance indicator.

18. A method for using a measurement device comprising the steps of:
a) providing the measurement device, wherein the measurement device further comprises:
a housing;
a sensor disposed in the housing to measure a force exerted on the housing and generate a pressure signal representing the force exerted on the housing; and
a digital display for receiving the pressure signal from the sensor and presenting a visual output to a user based upon the force exerted on the housing;
b) disposing the measurement device adjacent a portion of a body of a user;
c) exerting a first force on the housing of measurement device using the body of the user, wherein the display presents a first visual output representing the first force exerted on the housing; and
d) exerting a second force on the housing of measurement device using the body of the user, wherein the display presents a second visual output representing the second force exerted on the housing.

19. The method according to claim 17, wherein the visual output presented on the display is at least one of a pain indicator and a performance indicator.

20. The method according to claim 17, further comprising the step of:
e) comparing the first visual output and the second visual output to determine a change between the first force and the second force.
